(19) United States
(12) Patent Application Publication
(10) Pub. No.: US 2023/0272465 A1
(43) Pub. Date: Aug. 31, 2023

(54) Systems and Methods to Enhance RNA Transcription and Uses Thereof

(71) Applicant: The Board of Trustees of the Leland Stanford Junior University, Stanford, CA (US)

(72) Inventors: Eesha Sharma, Stanford, CA (US); Ivan Zheludev, Stanford, CA (US)

(73) Assignee: The Board of Trustees of the Leland Stanford Junior University, Stanford, CA (US)

(21) Appl. No.: 18/005,515

(22) PCT Filed: Jul. 13, 2021
(86) PCT No.: PCT/US21/41490

§ 371 Date: Jan. 13, 2023

Related U.S. Application Data
(60) Provisional application No. 63/051,271, filed on Jul. 13, 2020.

(51) Int. Cl. C12Q 1/6851; C12Q 1/686; C12N 9/12

(52) U.S. Cl. CPC C12Q 1/6851; C12Q 1/686; C12N 9/1247

(57) ABSTRACT

Turning now to the drawings, systems and methods to enhance RNA transcription in a cost-effective manner and uses thereof are provided. One of the most common enzymes for RNA transcription is T7 RNA polymerase. Many embodiments increase RNA yield in transcription reactions by adding ribonucleoside vanadyl complex (VRC) to the transcription reaction. Various embodiments use VRC at low concentrations in an RNA transcription reaction. Reactions in accordance with many embodiments are capable of increasing RNA yield by approximately 2-fold or more.

### CROSS-REFERENCE TO RELATED APPLICATIONS

The current application claims priority to U.S. Provisional Patent Application No. 63/051,271, filed Jul. 13, 2020; the disclosure of which is hereby incorporated by reference in its entirety.

### STATEMENT REGARDING FEDERALLY SPONSORED RESEARCH OR DEVELOPMENT

This invention was made with Governmental support under Contract No. 2RM1HG00773506 awarded by the National Institutes of Health. The government has certain rights in the invention.

### FIELD OF THE INVENTION

The present invention relates to ribonucleic acid (RNA) transcription. More specifically, the present invention relates to systems and methods to enhance RNA transcription by utilizing enzymatic inhibitors.

### BACKGROUND

Manufacturing RNA, including messenger RNA (mRNA), is very expensive. Typically, production of large quantities of RNA requires large quantities of reagents within transcription reactions. One of the most expensive components of the reaction mix is the enzyme used. At an industrial scale, the costs become exceptionally large, thus driving up the cost of downstream products, including vaccines, therapeutics, and other products. Currently, there is no cost-effective method or system to increase RNA production.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

In one embodiment, a method for increasing RNA transcription includes obtaining a nucleotide template, and transcribing RNA from the nucleotide template via an RNA transcription reaction including the nucleotide template, an RNA polymerase, nucleoside triphosphates, and ribonucleoside vanadyl complex (VRC).

In a further embodiment, the VRC is at a concentration of about 0.1 mM to about 10 mM.

In another embodiment, the VRC is at a concentration of about 0.1 mM.

In a still further embodiment, the VRC is at a concentration of about 1 mM.

In still another embodiment, the reaction is incubated at a temperature of approximately 20° C. to approximately 37° C.

In a yet further embodiment, the method further includes isolating the transcribed RNA.

In yet another embodiment, the isolating step comprises utilizing ethanol precipitation, isopropanol precipitation, column isolation, or DNase digestion.

In a further embodiment again, the method further includes quantifying the transcribed RNA.

In another embodiment again, the transcription reaction further includes a fluorescent dye; and the quantifying the RNA step comprises real-time monitoring of the reaction using a real-time thermal cycler.

In a further additional embodiment, the RNA polymerase is selected from: T7 RNA polymerase, Hi-T7® RNA polymerase, SP6 RNA polymerase, T3 RNA polymerase, E. coli RNA polymerase, RNA polymerase I, RNA polymerase II, and RNA polymerase III.

In another additional embodiment, the RNA polymerase is T7 RNA polymerase.

In a still yet further embodiment, the method further includes qualitatively analyzing the transcribed RNA.

In still yet another embodiment, the qualitatively analyzing step comprises utilizing agarose electrophoresis, polyacrylamide electrophoresis, or capillary electrophoresis.

In a still further embodiment again, a kit for transcribing RNA includes an RNA polymerase, nucleoside triphosphates, ribonucleoside vanadyl complex (VRC), and a buffer.

In still another embodiment again, the RNA polymerase is selected from T7 RNA polymerase, Hi-T7® RNA polymerase, SP6 RNA polymerase, T3 RNA polymerase, E. coli RNA polymerase, RNA polymerase I, RNA polymerase II, and RNA polymerase III.

In a still further additional embodiment, the RNA polymerase is T7 RNA polymerase.

In still another additional embodiment, the kit further includes nuclease-free water.

In a yet further embodiment again, the RNA polymerase, the nucleoside triphosphates, the VRC, and the buffer are provided as a lyophilized tablet.

Other features and advantages of the present invention will become apparent from the following detailed description, taken in conjunction with the accompanying drawings which illustrate, by way of example, the principles of the invention.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIGS. 1A-1B illustrate line graphs illustrating RNA yield in RNA transcription reactions in real time in accordance with various embodiments of the invention.

FIG. 2 illustrates a bar graph illustrating increased RNA yield in RNA transcription reactions in accordance with various embodiments of the invention.

FIG. 3 illustrates results of gel electrophoresis of RNA transcription reactions in accordance with various embodiments of the invention.

FIG. 4 illustrates an exemplary method to transcribe RNA in accordance with various embodiments of the invention.

### DETAILED DESCRIPTION

Turning now to the drawings, systems and methods to enhance RNA transcription in a cost-effective manner and uses thereof are provided. Many embodiments increase RNA yield in transcription reactions by adding a nuclease inhibitor to a transcription reaction. Various embodiments utilize ribonucleoside vanadyl complex (VRC) as the nuclease inhibitor. Various embodiments use VRC at relatively low concentrations in an RNA transcription reaction. Reactions in accordance with many embodiments are capable of increasing RNA yield by approximately 2-fold or more.

VRC is a potent inhibitor of many nucleic acid modifying enzymes, including ribonucleases, transcriptases, polymerases, phosphatases, and ligases. (See e.g., Berger, S. L. and Birkenmeier, C. S. (1979). Biochemistry. 18; Gray, J. C. (1974). Arch. Biochem. Biophys. 163, 343-348; Egberts, E., Hackett, P. B. and Traub, P. (1971). Hoppe-Zeyler's Z. Physiol Chem. 358, 475-490; Puskas, R. S., et al. (1982). Biochemistry 1982, 21, 19, 4602-4608; and Berger, S. L., et al. (1980). J. Biol. Chem. 255, 7, 2955-61; the disclosures of which are hereby incorporated by reference herein in their entireties.) Furthermore, VRC is a relatively inexpensive reagent, thus providing increased RNA yield without a significant increased cost.

One of the most common enzymes for RNA transcription is T7 RNA polymerase. Many commercially available kits exist that have been optimized to provide high yields of RNA from T7 RNA polymerase reactions. However, much many additional polymerases can be used for RNA transcription, including (but not limited to) SP6 RNA polymerase, T3 RNA polymerase, E. coli RNA polymerase, RNA polymerase I, RNA polymerase II, RNA polymerase III, and/or any other relevant RNA polymerase.

Many embodiments provide a nuclease inhibitor (e.g., VRC) to enhance RNA transcription with one or more of the RNA polymerases noted above. Certain embodiments utilize VRC at concentrations from about 0.1 mM (±0.05 mM) to about 10 mM (±2 mM). Under these conditions, various embodiments add a nominal cost of approximately $0.000086 per 20 μL reaction volume.

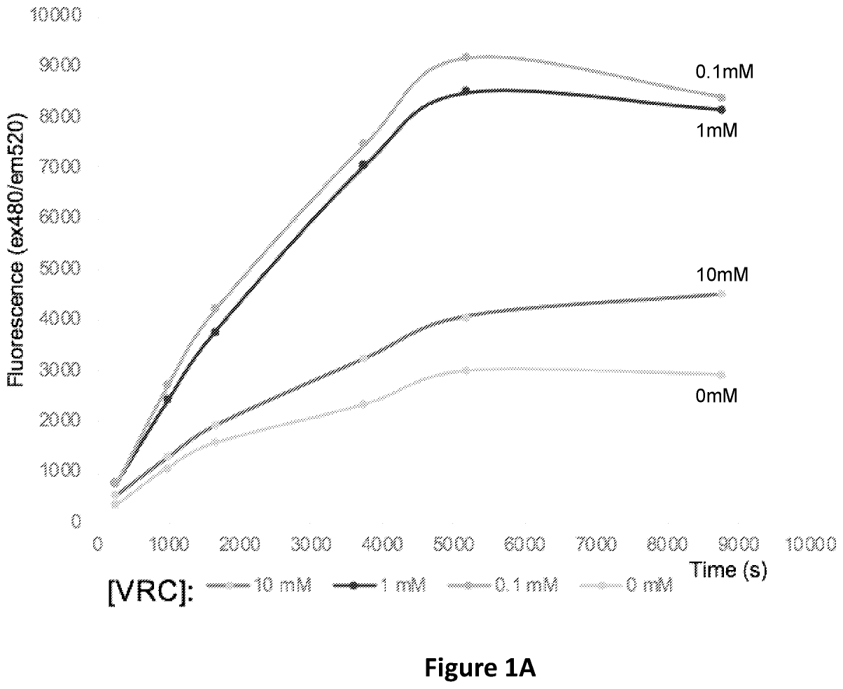

Turning to FIGS. 1A-1B, exemplary data of RNA yield measured from real time monitoring of RNA transcription reactions in accordance with certain embodiments are illustrated. In particular, FIG. 1A illustrates exemplary T7 RNA polymerase transcription reaction embodiments using 0.1 mM, 1 mM, and 10 mM concentrations of VRC against a 0 mM control. As illustrated, embodiments using from about 0.1 mM to 1 mM VRC exhibit about 2.7-fold increase in yield of RNA, while embodiments using 10 mM VRC exhibit approximately a 1.5-fold increase in RNA yield.

However, FIG. 1B illustrates exemplary SP6 RNA polymerase transcription reaction embodiments using 0.1 mM, 1 mM, and 10 mM concentrations of VRC against a 0 mM control. As illustrated in this exemplary data, at 0.1 mM concentrations, VRC shows no advantage in yield over the control. Additionally, at concentrations of 1 mM and 10 mM, VRC appears to show inhibition of SP6 RNA polymerase, akin to known inhibition regarding various enzymes, including reverse transcriptase and nucleases.

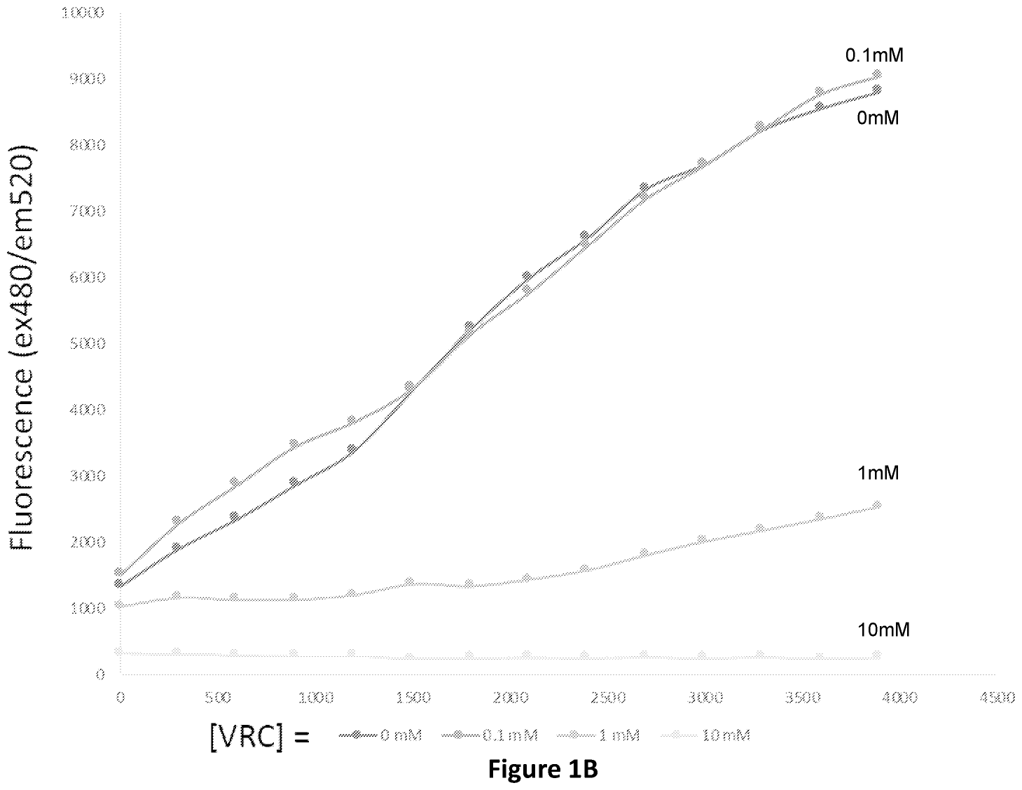

Turning to FIG. 2, RNA yield measured post-reaction is illustrated in accordance with certain embodiments. Similar to the exemplary results in FIG. 1A, FIG. 2 illustrates exemplary results of a reaction in accordance with certain embodiments, where the exemplary embodiment using 0.1 mM VRC possesses an approximately 2.3-fold increase in RNA yield, while the exemplary embodiment using 1 mM VRC illustrates possesses an approximately 1.7-fold increase in RNA yield and the exemplary embodiment using 10 mM VRC illustrates possesses an approximately 1.5-fold increase in RNA yield.

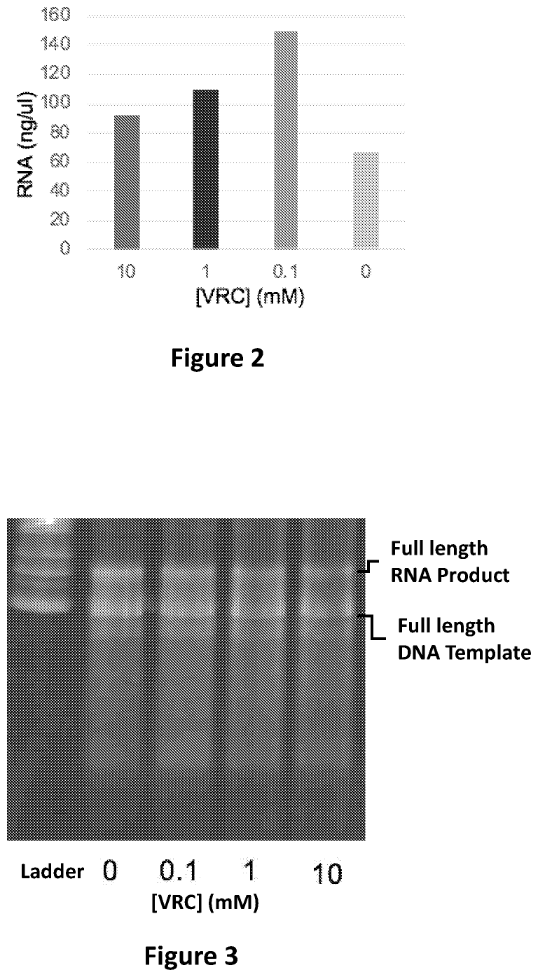

Turning to FIG. 3, a gel of reaction products of various exemplary embodiments showing that there is no degradation of products, thus showing that VRC is responsible for the increased RNA yield in many embodiments. Specifically, FIG. 3 illustrates clear bands for full length RNA product and full length template in the exemplary 0.1 mM, 1 mM, and 10 mM embodiments and 0 mM control. Since there is no indication of degradation products or changes in degradation products, FIG. 3 indicates that VRC is directly responsible for the increases in yield by T7 RNA polymerase, rather than by inhibiting nucleases or other enzymes in the reaction conditions.

### Methods

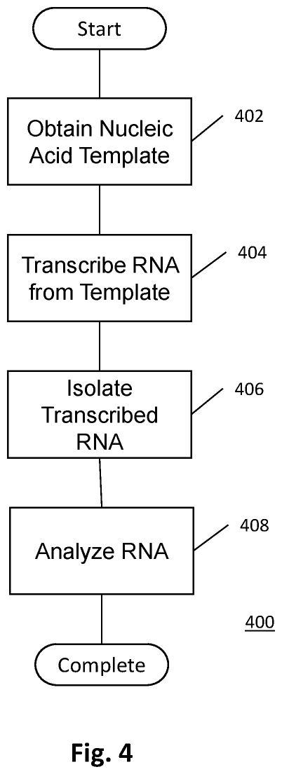

Many embodiments are directed to methods of transcribing RNA. FIG. 4 illustrates an exemplary method 400 for enhancing RNA transcription using a nuclease inhibitor. At 402, many embodiments obtain a nucleic acid template. The nucleic acid template can be DNA, RNA, a template possessing both DNA and RNA, or combinations thereof. In some embodiments, the template comprises a coding sequence of a gene. In certain embodiments, the template further comprises additional components for RNA function, including (but not limited to) a 5′ untranslated regions (UTR), a 3′UTR, polyA tail, a polyA tailing sequence, an indexing sequence (e.g., a barcode sequence), and/or any other feature that can be incorporated within an RNA construct for RNA function. Some embodiments obtain a pool of templates, where the pool comprises multiple unique template sequences. In some embodiments of pooled templates, each unique RNA sequence comprises a unique indexing sequence.

In certain embodiments, the template is sequence optimized for a particular RNA structure and/or codon optimized for preferred codons for a particular species. Methods are known in the art to codon optimize or alter sequences to create structure in an RNA molecule.

At 404, several embodiments transcribe RNA from the template. In various embodiments, transcribing RNA utilizes a transcription reaction. In various embodiments, the transcription reaction comprises an RNA polymerase. Depending on the template molecule (e.g., DNA or RNA), the RNA polymerase is selected from an RNA-dependent RNA polymerase or a DNA-dependent RNA polymerase. Various embodiments select the RNA polymerase from T7 RNA polymerase, Hi-T7® RNA polymerase, SP6 RNA polymerase, T3 RNA polymerase, E. coli RNA polymerase, RNA polymerase I, RNA polymerase II, and RNA polymerase III.

Many embodiments further comprise a nuclease inhibitor in the transcription reaction. In many embodiments, the nuclease inhibitor is VRC. Various embodiments provide VRC at a concentration of approximately 0.1 mM to approximately 10 mM, including approximately 0.1 mM, approximately 1 mM, and approximately 10 mM.

Further reactions include relevant nucleoside triphosphates (NTPs), including ATP, UTP, GTP, and/or CTP for the reaction. Additional embodiments include additional components to assist in RNA transcription, such as buffers, one or more primers, DMSO, salts, dyes, and/or any other compound or reagent. Reaction profile (e.g., temperature cycling) in various embodiments can be adjusted depending on the selected enzyme, template length, and/or primer melting temperature, as known in the art. In various embodiments, the reaction is incubated at a temperature of approximately 20° C. to about 37° C. (±5° C.). In certain embodiments, the reaction is incubated for approximately 30 minutes to approximately 6 hours.

Additional embodiments isolate transcribed RNA at 406. RNA isolation in accordance with various embodiments can include various methods known in the art, including alcohol precipitation (including ethanol precipitator and isopropanol precipitation), column isolation, DNase digestion, and/or any other method known in the art. Certain embodiments resuspend the isolated RNA in a solution, such as nuclease-free water or buffer.

Further embodiments analyze the RNA at 408. RNA analysis in accordance with embodiments can include quantitative and/or qualitative analysis. Certain embodiments utilize UV-Vis spectroscopy to quantify RNA within a solution, while some embodiments utilize fluorescent dyes or probes and fluorescence to quantify RNA concentration. Certain embodiments qualitatively analyze RNA to verify RNA transcription, such as full-length RNA transcription. Various embodiments utilize qualitative (e.g., agarose and polyacrylamide) or capillary electrophoresis to verify amplification. Certain embodiments are capable of providing the quantitative and qualitative analysis simultaneously, such as through quantitative electrophoresis, including gel or capillary electrophoresis to quantify RNA concentration and verify full-length RNA transcription.

It should be noted that various features of method 400 may be perfumed multiple times, omitted, completed in a different order, or completed simultaneously than as described in relation to FIG. 4. For example, some embodiments may analyze RNA 408 (e.g., quantify RNA) simultaneously with transcribing RNA 404, such as through the use of a fluorescent dye and a real time thermal cycler to monitor the reaction. Furthermore, some embodiments may omit quantitative or qualitative analysis altogether.

### Kits

In several embodiments, kits are utilized for transcribing RNA. Kits in accordance with various embodiments may include one or more reagents to transcribe RNA and printed instructions for transcribing RNA. The reagents may be packaged in separate containers. However, in some kits, the reagents are packaged as a single component ready to start a reaction. In some embodiments, the reaction components are prepackaged in reaction tubes in either liquid or lyophilized form, such that a nucleic acid template can be added directly with or without additional liquid (e.g., water) to fill reaction components to a specific concentration or final volume. In certain embodiments, the kits provide the reagents in a lyophilized tablet or lozenge that can be added to a separate reaction vessel (e.g., reaction tube, test tube, reaction plate, etc.).

In addition to RNA reactions to increase yield using VRC, further embodiments are directed to kits including components and reagents for increased RNA production from T7 RNA polymerase. As such, many embodiments of kits include reagents to transcribe RNA in addition to VRC. In certain embodiments the kit includes VRC and one or more of the following reagents: reaction buffer, nuclease-free water, nucleoside triphosphates (NTPs) (e.g., ATP, GTP, CTP, and UTP), an RNA polymerase (e.g., T7 RNA polymerase, SP6 RNA polymerase), spermine, spermidine, DMSO, and/or any other component that can be used for RNA transcription.

A kit can include suitable containers for the reagents and/or reaction including, for example, bottles, vials, syringes, and test tubes. Containers can be formed from a variety of materials, including glass or plastic. The kit can also comprise a package insert containing written instructions for methods of detecting one or more target nucleic acids.

### Exemplary Embodiments

Although the following embodiments provide details on certain embodiments of the inventions, it should be understood that these are only exemplary in nature, and are not intended to limit the scope of the invention.

### Example 1: Real-Time RNA Transcription

Methods: A series of standard RNA transcription reactions were performed using T7 polymerase spiked with 0 mM, 0.1 mM, 1 mM, and 10 mM VRC. The reaction was performed at room temperature (˜20° C.) for 9000 seconds in a TECAN plate reader. Reaction yield was measured in reach reaction by measuring fluorescence in real-time using the TECAN plate reader.

Results: In FIG. 1A, reactions using 0.1 mM to 10 mM VRC produced increased yield over the control, 0 mM VRC, reaction. Specifically, the 0.1 mM and 1 mM VRC reactions produced an approximately 2.7-fold increase in RNA yield, while the 10 mM VRC reaction produced approximately a 1.5-fold increase in RNA yield. This pattern is illustrated across the entirety of the time course.

Conclusion: Including VRC in an RNA transcription reaction produces increased yield throughout an entire RNA transcription reaction. Additionally, the highest yield may occur in a specific range of VRC concentrations.

### Example 2: RNA Yield after Post-Reaction Cleaning

Methods: A series of standard RNA transcription reactions were performed using T7 polymerase spiked with 0 mM, 0.1 mM, 1 mM, and 10 mM VRC. The reaction was performed at 37° C. for 4 hours in a thermocycler. A post-reaction clean-up was used to remove any reagents, including enzymes, NTPs, buffers. The reaction was quantified using a NanoDrop UV-Vis spectrometer.

Results: In FIG. 2, reactions using 0.1 mM to 10 mM VRC produced increased yield over the control, 0 mM VRC, reaction. Specifically, the 0.1 mM VRC reaction produced an approximately 2.3-fold increase in RNA yield, while the exemplary embodiment using 1 mM VRC illustrates possesses an approximately 1.7-fold increase in RNA yield and the exemplary embodiment using 10 mM VRC illustrates possesses an approximately 1.5-fold increase in RNA yield.

Conclusion: Including VRC in an RNA transcription reaction produces increased yield, even after any potential off-products were removed. Additionally, the highest yield may occur in a specific range of VRC concentrations.

### Example 3: VRC Enhances Transcription

Methods: A series of standard RNA transcription reactions were performed using T7 polymerase spiked with 0 mM, 0.1 mM, 1 mM, and 10 mM VRC. Post-incubation, the contents of each reaction were electrophoresed on a gel to qualitatively identify reaction product sizes.

Results: In FIG. 3, the reaction products appear identical across each reaction condition (0 mM, 0.1 mM, 1 mM 10 mM VRC) with no discernable differences in product sizes.

Conclusion: Since there is no indication of degradation products or changes in degradation products, coupled with the kinetic data in FIG. 1A, VRC appears directly responsible for the increases in yield by T7 RNA polymerase, rather than by inhibiting nucleases or other enzymes in the reaction conditions.

### DOCTRINE OF EQUIVALENTS

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

What is claimed is:

1. A method for increasing RNA transcription comprising:
obtaining a nucleotide template; and
transcribing RNA from the nucleotide template via an RNA transcription reaction comprising the nucleotide template, an RNA polymerase, nucleoside triphosphates, and ribonucleoside vanadyl complex (VRC).

2. The method of claim 1, wherein the VRC is at a concentration of about 0.1 mM to about 10 mM.

3. The method of claim 1, wherein the VRC is at a concentration of about 0.1 mM.

4. The method of claim 1, wherein the VRC is at a concentration of about 1 mM.

5. The method of claim 1, wherein the reaction is incubated at a temperature of approximately 20° C. to approximately 37° C.

6. The method of claim 1, further comprising isolating the transcribed RNA.

7. The method of claim 6, wherein the isolating step comprises utilizing ethanol precipitation, isopropanol precipitation, column isolation, or DNase digestion.

8. The method of claim 1, further comprising quantifying the transcribed RNA.

9. The method of claim 8, wherein the transcription reaction further comprises a fluorescent dye; and wherein the quantifying the RNA step comprises real-time monitoring of the reaction using a real-time thermal cycler.

10. The method of claim 1, wherein the RNA polymerase is selected from: T7 RNA polymerase, Hi-T7® RNA polymerase, SP6 RNA polymerase, T3 RNA polymerase, E. coli RNA polymerase, RNA polymerase I, RNA polymerase II, and RNA polymerase III.

11. The method of claim 1, wherein the RNA polymerase is T7 RNA polymerase.

12. The method of claim 1, further comprising qualitatively analyzing the transcribed RNA.

13. The method of claim 12, wherein the qualitatively analyzing step comprises utilizing agarose electrophoresis, polyacrylamide electrophoresis, or capillary electrophoresis.

14. A kit for transcribing RNA comprising:
an RNA polymerase;
nucleoside triphosphates;
ribonucleoside vanadyl complex (VRC); and
a buffer.

15. The kit of claim 14, wherein the RNA polymerase is selected from: T7 RNA polymerase, Hi-T7® RNA polymerase, SP6 RNA polymerase, T3 RNA polymerase, E. coli RNA polymerase, RNA polymerase I, RNA polymerase II, and RNA polymerase III.

16. The kit of claim 14, wherein the RNA polymerase is T7 RNA polymerase.

17. The kit of claim 14, further comprising nuclease-free water.

18. The kit of claim 14, wherein the RNA polymerase, the nucleoside triphosphates, the VRC, and the buffer are provided as a lyophilized tablet.
